(19) United States
(12) Patent Application Publication
(10) Pub. No.: US 2005/0113751 A1
(43) Pub. Date: May 26, 2005

(54) Safety syringe capable of avoiding retraction of a needle during sucking medicine/injection

(76) Inventor: Fu-Yee Chen, San -Choung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/972,353

(22) Filed: Oct. 26, 2004

(30) Foreign Application Priority Data
Nov. 7, 2003 (TW) 092219777

(51) Int. Cl. A61M 5/00

(52) U.S. Cl. 604/110; 604/198

(57) ABSTRACT

A safety syringe capable of avoiding retraction of a needle during sucking medicine/injection, including: a barrel; a needle holder inserted in the top end of the barrel for holding the needle; a first leakproof body fitted on the bottom end of the needle holder, a circumference of the first leakproof body tightly attaching to the inner wall of the top end of the barrel; and a plunger slidably fitted in the barrel, the top end of the plunger being formed with a latch head section for correspondingly latching with the needle holder. A circumference of the bottom end of the needle holder is formed with multiple flange sections. An annular fixing groove is formed on top end of the barrel. The flange sections of the needle holder are inlaid in the annular fixing groove, to prevent the needle be retreated into the barrel during sucking or injection.

### BACKGROUND OF THE INVENTION

1. Field of the Invention

The present invention is related to a safety syringe capable of avoiding retraction of a needle during sucking medicine/injection. The outer circumference of bottom end of the needle holder is formed with multiple flange sections. An annular fixing groove is formed on top end of the barrel. The flange sections of the needle holder are correspondingly inlaid in the annular fixing groove. When thrusting the needle into a vial for sucking a medicine or during injection, the needle is prevented from being retreated into the barrel by the reaction force.

2. Description of the Prior Art

It is known that the main infectious medium of acquired immunodeficiency syndrome (AIDS) is blood. Medical workers or those persons dealing with medical wastes often need to contact with waste syringes. Therefore, the possibility of being impaled by the waste needle for these people is greater than any other person. As a result, these medical workers take greater chance of getting infected with AIDS. A safety syringe has been developed to avoid this problem. After injection, the needle can be tracked back into the barrel. Then the plunger is again pushed back to bend and deform the needle. Therefore, after injection, the needle is retracted in the barrel without protruding outward. This avoids impalement of medical workers. However, the current safety syringe still has some shortcomings as follows:

1. There is no reliable temporarily fixing structure between the needle and the barrel. When the needle is thrust into the cork of the vial for sucking the medicine or when the needle is thrust through a patient's skin for injection, the resistance often makes the needle retreated. At this time, the needle is no more tightly fixed at the front end of the barrel and is retracted into the barrel. This leads to failure of injection.

2. When the plunger pushes back the needle to bend the same, the needle is pressed against the wall of the plastic-made barrel. Therefore, it often takes place that the needle thrusts through the barrel. It is troublesome to deal with such situation.

3. The needle can be hardly tightly connected with the barrel so that the liquid medicine often leaks through the gap between the needle and the barrel.

### SUMMARY OF THE INVENTION

It is therefore a primary object of the present invention to provide a safety syringe capable of avoiding retraction of a needle during sucking medicine/injection, including: a barrel; a needle holder inserted in top end of the barrel for holding the needle; a first leakproof body fitted on bottom end of the needle holder, a circumference of the first leakproof body tightly attaching to inner wall of the top end of the barrel; and a plunger slidably fitted in the barrel, top end of the plunger being formed with a latch head section for correspondingly latching with the needle holder. When the plunger downward pulls the needle holder, the needle held by the needle holder is pulled into the barrel. A circumference of bottom end of the needle holder is formed with multiple flange sections. An annular fixing groove is formed on top end of the barrel. The flange sections of the needle holder are inlaid in the annular fixing groove, whereby when thrusting the needle into a vial for sucking a medicine or during injection, the needle is prevented from being retreated into the barrel.

The present invention can be best understood through the following description and accompanying drawings wherein:

### BRIEF DESCRIPTION OF THE DRAWINGS

FIG. 1 is a sectional view of the present invention free from any needle;

FIG. 2 is a sectional view of the present invention connected with a needle;

FIG. 3 is a sectional view taken along line 3-3 of FIG. 1;

FIG. 4 is a top view of the needle holder of the present invention;

FIG. 5 is a sectional view of the present invention after injection;

FIG. 6 is a sectional view according to FIG. 5, in which the needle holder is pulled back;

FIG. 7 is a sectional view of the present invention, in which the plunger is bent but not yet broken; and

FIG. 8 is a sectional view according to FIG. 7, in which the plunger is broken.

### DETAILED DESCRIPTION OF THE PREFERRED EMBODIMENTS

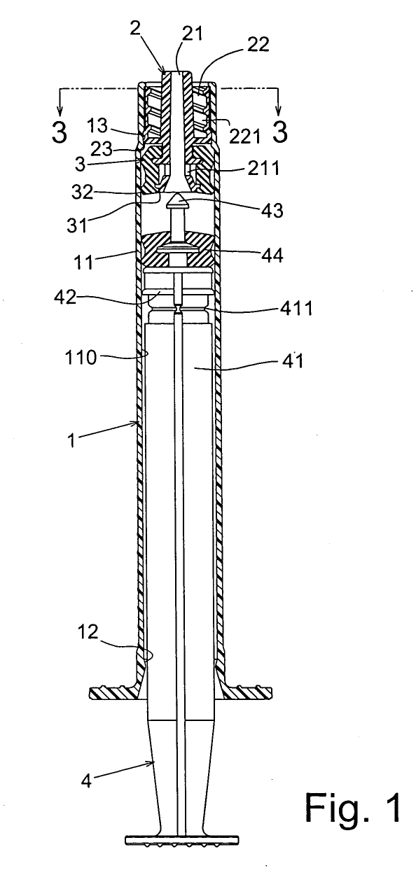

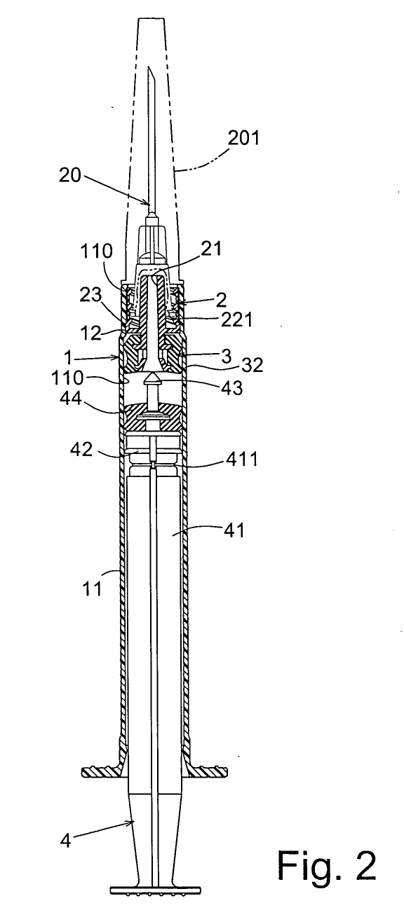

Please refer to FIGS. 1 to 8. The safety syringe of the present invention includes: a barrel 1; a needle holder 2 inserted in top end of the barrel 1 for holding a needle 20; a first leakproof body 3 fitted on bottom end of the needle holder 2, a circumference of the first leakproof body 3 tightly attaching to inner wall of the top end of the barrel 1; and a plunger 4 slidably fitted in the barrel 1. The top end of the plunger 4 is formed with a latch head section 43 for latching with the needle holder 2. When the plunger 4 downward pulls the needle holder 2, the needle 20 held by the needle holder 2 is also pulled into the barrel 1.

The barrel 1 has a barrel body 11. A stopper ring 12 inward protrudes from the inner wall 110 of the barrel body 11 near bottom end thereof. The stopper ring 12 serves to abut against the bottom edge of the front head section 42 of the plunger 4 so as to stop the plunger 4 from being totally extracted out of the bottom end of the barrel 1. In addition, an annular fixing groove 13 is formed on the inner wall 110 of the top end of the barrel 1, whereby a flange section 23 of the bottom end of the needle holder 2 is inlaid in the annular fixing groove 13. Accordingly, when thrusting the needle into the vial or in injection, the resistance against the needle cannot forcedly retreat the needle holder 2 into the barrel 1. Therefore, the safety syringe of the present invention can be normally used.

Referring to FIGS. 1 to 3, the needle holder 2 is formed with an axial central through hole 21. A radial latch hole 211 is formed near the bottom end of the central through hole 21, whereby the latch head section 43 of the front end of the plunger 4 can be correspondingly tightly latched in the latch hole 211. The top section of the needle holder 2 is formed with a fitting cavity 22. A thread 221 is formed on the wall of the fitting cavity 22, whereby a bottom section of the needle 20 can be screwed into the fitting cavity 22 to prevent the needle 20 from detaching from the needle holder 2.

Referring to FIG. 4, the circumference of the bottom end of the needle holder 2 is formed with multiple flange sections 23 which can be arched flanges. The shape of the flanges 23 is not limited.

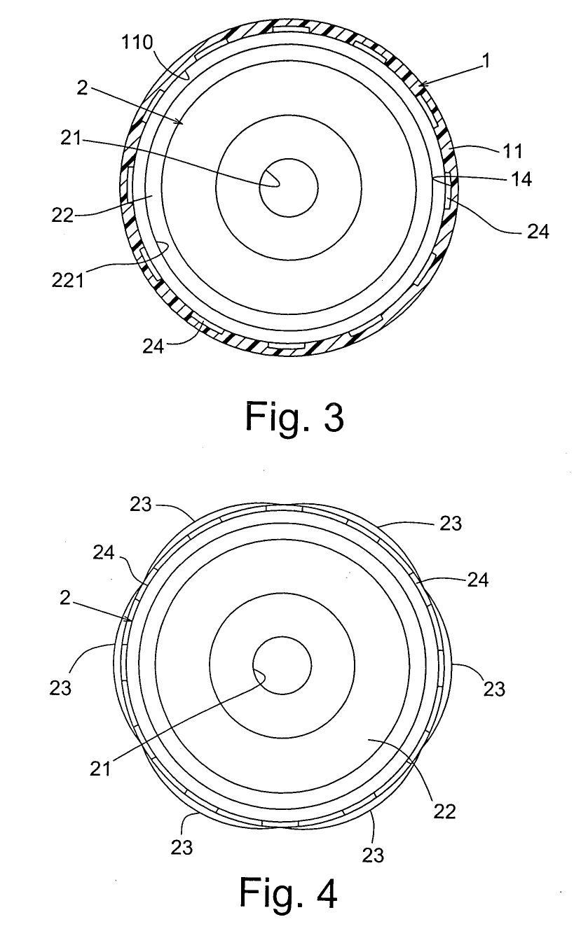

Referring to FIGS. 3 and 4, multiple guide ribs 24 are formed on the outer circumference of the top section of the needle holder 2 at equal angular intervals. The wall of top end of the barrel 1 is formed with guide channels 14 corresponding to the guide ribs 24. The guide ribs 24 can be slid into the guide channels 14, whereby the needle holder 2 can be guided and slid into the barrel 1.

The first leakproof body 3 has a fitting dent 31 for correspondingly tightly fitting on the bottom section of the needle holder 2. The outer circumference 32 of the first leakproof body 3 tightly abuts against the inner wall of the barrel 1 so as to prevent the liquid medicine filled in the barrel 1 from leaking out from the top end of the barrel 1.

The plunger 4 has a stem body 41 and a head section 42 connected with front end of the stem 41. The latch head section 43 axially projects from front end of the head section 42 for correspondingly latching with the bottom end of the needle holder 2. A second leakproof body 44 is fitted around the head section 42. The outer circumference of the second leakproof body 44 tightly attaches to the inner wall 110 of the barrel 1 so as to prevent the liquid medicine filled in the barrel 1 from leaking out from the bottom section of the barrel 1.

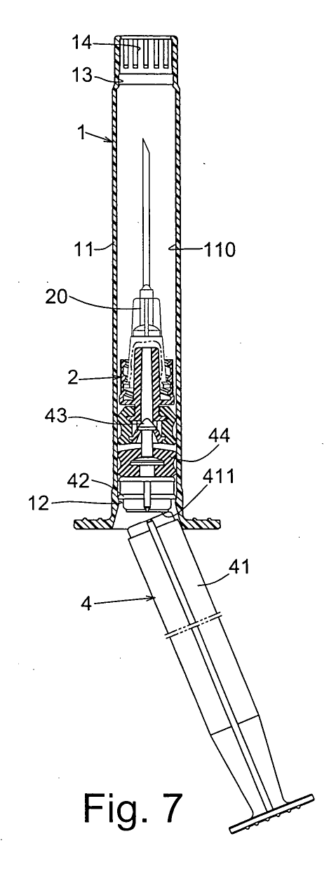

The circumference of the front end of the stem body 41 of the plunger 4 is formed with a breaking groove 411. After the plunger 4 pulls back the needle holder 2 and the needle 20 is driven by the needle holder 2 to slide into the barrel 1, as shown in FIG. 7, the plunger 4 can be bent to one side. At this time, the stress is concentrated on the breaking groove 411 to break the stem body 41. After broken, the plunger 4 remains and sticks in the barrel 1 without protruding from the barrel 1.

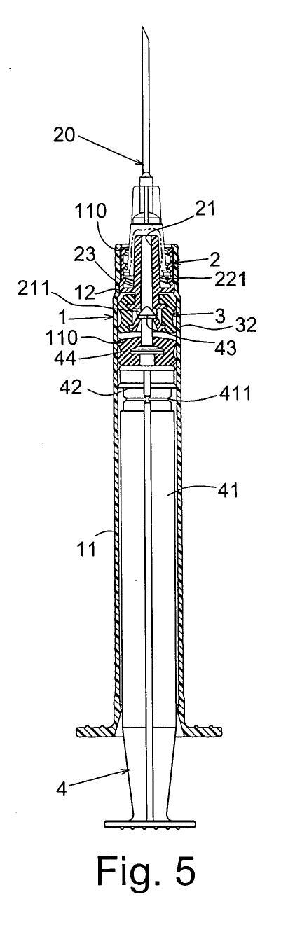

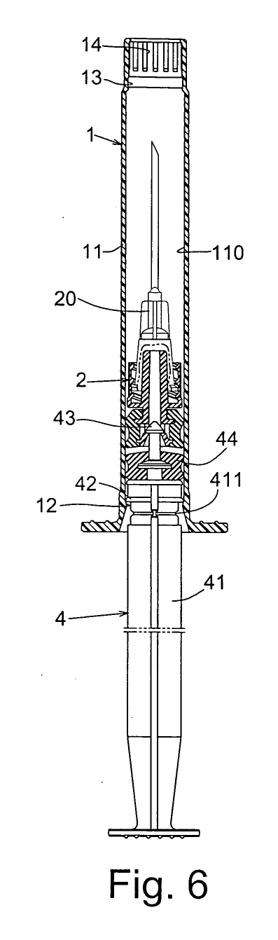

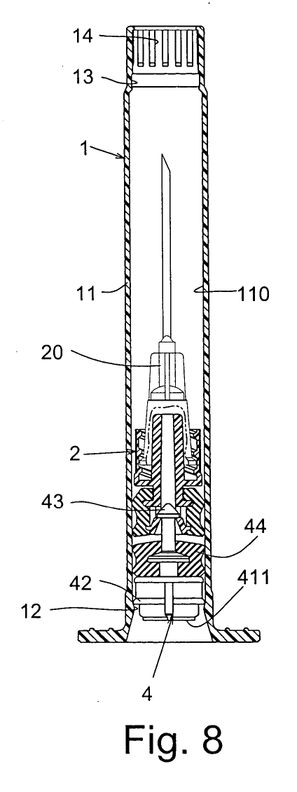

Referring to FIG. 1, when released from the factory, the needle holder 2 has been already inserted in the top end of the barrel 1. Also, the flange sections 23 of the bottom end of the needle holder 2 are inlaid in the annular fixing groove 13 of the top end of the barrel 1. The top end of the plunger 4 is positioned in the barrel 1. The latch head section 43 of the front end of the plunger 4 has not yet latched with the needle holder 2. When used, a medical worker can screw a needle 20 into the fitting cavity 22 of the top section of the needle holder 2. After the protective jacket 201 of the needle 20 is taken off, the needle 20 can be thrust into a vial for sucking the medicine. As shown in FIG. 5, after injection, the latch head section 43 of the plunger 4 is correspondingly inserted into the latch hole 211 of the bottom end of the needle holder 2. Referring to FIG. 6, when the medical worker pulls down the plunger 4, the plunger 4 drivingly tracks down the needle holder 2. At this time, the flange sections 23 of the needle holder 2 are detached from the annular fixing groove 13 of the barrel 1 and the needle holder 2 together with the needle 20 is pulled back into the barrel 1. When the plunger 4 is further moved downward, the circumference of the bottom of the head section 42 of the plunger 4 abuts against the stopper ring 12 of the bottom end of the barrel 1. Under such circumstance, the plunger 4 is stopped from further moving downward. At this time, the needle 20 is totally retracted into the barrel 1 without protruding outward to hurt anyone. Referring to FIG. 7, the medical worker can bend the stem body 41 of the plunger 4 left and right to break the stem body 41. As shown in FIG. 8, after broken, the remainder of the plunger 4 remains and sticks in the barrel 1 without protruding from the barrel 1. Accordingly, it is impossible to further push the plunger 4 and protrude the needle 20 from the barrel 1. This ensures the safety of the medical workers.

According to the above arrangement, the safety syringe capable of avoiding retraction of the needle during sucking medicine/injection of the present invention has the following advantages:

1. The circumference of the bottom end of the needle holder 2 is formed with the flange sections 23 for temporarily tightly latching the needle holder 2 with the barrel 1. Therefore, when thrusting the needle 20 into the vial or during injection, the reaction force against the needle cannot forcedly retreat the needle holder 2 into the barrel 1. Therefore, the injection can be completed successfully.

2. The first leakproof body 3 is fitted on the bottom section of the needle holder 2 to effectively prevent the liquid medicine filled in the barrel 1 from leaking out.

3. The needle holder 2 is formed with a fitting cavity 22. A thread 221 is formed on the wall of the fitting cavity 22. The needle 20 can be screwed into and reliably fixed in the fitting cavity 22 of the needle holder 2 to prevent the needle 20 from detaching from the needle holder 2.

The above embodiments are only used to illustrate the present invention, not intended to limit the scope thereof. Many modifications of the above embodiments can be made without departing from the spirit of the present invention.

1. A safety syringe capable of avoiding retraction of a needle during sucking medicine/injection, comprising:
(a) a barrel;
(b) a needle holder inserted in top end of the barrel for holding the needle;
(c) a first leakproof body fitted on bottom end of the needle holder, a circumference of the first leakproof body tightly attaching to inner wall of the top end of the barrel; and
(d) a plunger slidably fitted in the barrel, top end of the plunger being formed with a latch head section for correspondingly latching with the needle holder, whereby when the plunger downward pulls the needle holder, the needle held by the needle holder is pulled into the barrel, a circumference of bottom end of the needle holder being formed with multiple flange sections, an annular fixing groove being formed on top end of the barrel, the flange sections of the needle holder being inlaid in the annular fixing groove, whereby when thrusting the needle into a vial for sucking a medicine or during injection, the needle is prevented from being retreated into the barrel.

2. The safety syringe capable of avoiding retraction of a needle during sucking medicine/injection as claimed in claim 1, wherein the barrel has a barrel body, a stopper ring inward protruding from inner wall of the barrel body near bottom end thereof, the stopper ring serving to abut against bottom edge of a front head section of the plunger so as to stop the plunger from being totally extracted out of the bottom end of the barrel, the annular fixing groove being formed on the inner wall of the top end of the barrel, whereby the flange sections of the bottom end of the needle holder are snugly inlaid in the annular fixing groove.

3. The safety syringe capable of avoiding retraction of a needle during sucking medicine/injection as claimed in claim 1, wherein the needle holder is formed with an axial central through hole, a radial latch hole being formed near bottom end of the central through hole, whereby the latch head section of the front end of the plunger can be correspondingly tightly latched in the latch hole, a top section of the needle holder being formed with a fitting cavity, a thread being formed on a wall of the fitting cavity, whereby a bottom section of the needle can be screwed into the fitting cavity.

4. The safety syringe capable of avoiding retraction of a needle during sucking medicine/injection as claimed in claim 1, wherein the flange sections of the needle holder are arched flanges.

5. The safety syringe capable of avoiding retraction of a needle during sucking medicine/injection as claimed in claim 1, wherein multiple guide ribs are formed on outer circumference of the top section of the needle holder at equal angular intervals, a wall of top end of the barrel being formed with guide channels corresponding to the guide ribs, whereby the guide ribs can be slid into the guide channels.
